(19) United States
(12) Patent Application Publication
(10) Pub. No.: US 2011/0288545 A1
(43) Pub. Date: Nov. 24, 2011

(54) Method and Device for Ablation of Cancer and Resistance to New Cancer Growth

(75) Inventors: Stephen J. Beebe, Norfolk, VA (US); Xinhua Chen, Norfolk, VA (US); Karl H. Schoenbach, Norfolk, VA (US)

(73) Assignee: OLD DOMINION UNIVERSITY RESEARCH FOUNDATION, Norfolk, VA (US)

(21) Appl. No.: 13/092,518

(22) Filed: Apr. 22, 2011

Related U.S. Application Data
(60) Provisional application No. 61/326,851, filed on Apr. 22, 2010.

(51) Int. Cl. A61B 18/18

(52) U.S. Cl. 606/41

(57) ABSTRACT

Methods and devices designed to eliminate and/or ablate cancer or other abnormal growths of cells or tissues or eliminate and/or ablate cells or tissue with abnormal functions. In particular, using sub-microsecond electric pulses to treat cancer cells by, inter alia, inducing programmed cell death or other type of cell death. These methods and devices are expected to greatly improve the prevention, treatment and management of cancer by increasing the effectiveness of cancer treatments and development of resistance to new cancer growth.

### CROSS-REFERENCE TO RELATED APPLICATIONS

This application claims priority to U.S. Provisional Application No. 61/326,851, entitled “Method and Device for Ablation of Cancer and Resistance to New Cancer Growth,” filed Apr. 22, 2010, the entirety of which is incorporated herein by reference.

### FIELD OF THE INVENTION

The invention relates generally to the fields of molecular biology and cellular biology.

### BACKGROUND

Cancer is a serious human health concern and a leading cause of death worldwide. According to the World Health Organization, deaths from cancer worldwide are projected to continue rising, with an estimated 12 million deaths in 2030. In the United States (U.S.), cancer is the second leading cause of death. The National institutes of Health estimates overall costs of cancer in 2008 at $228.1 billion: $93.2 billion for direct medical costs (total of all health expenditures); $18.8 billion for indirect morbidity costs (cost of lost productivity due to illness); and $116.1 billion for indirect mortality costs (cost of lost productivity due to premature death).

Therefore, developing effective methods for the prevention, treatment and management of cancer is urgently required. Traditional cancer treatments have included combinations of surgery, chemotherapy and radiotherapy and vary depending on the specific type, location of the tumor and stage of the disease. However, the ability of tumor cells to evade engagement of apoptosis can play a significant role in their resistance to traditional treatments.

### SUMMARY

The methods and devices described herein provide treatments for the elimination and/or ablation of cancer by programmed cell death and other types of cell death through the application of nanosecond pulsed electric fields (nsPEF). These methods and devices are expected to greatly improve the prevention, treatment and management of cancer by increasing the effectiveness of cancer treatments and development of resistance to new cancer growth. The methods and devices described herein provide a solution to the problems associated with conventional methods (e.g., the ability of cancer cells to evade apoptosis and other types of cell death) by inducing programmed and other types of cell death.

Unless otherwise defined, all technical terms used herein have the same meaning as commonly understood by one of ordinary skill in the art to which this invention belongs.

As used herein, “cancer” and “cancer cells” include any type of cancer and any cell or tissue with abnormal functions, and is not limited to any particular type of cancer.

By the term “sub-microsecond” is meant a duration less than one microsecond, including without limitation 999 nanoseconds (ns) or less.

Although methods and devices similar or equivalent to those described herein can be used in the practice or testing of the present invention, suitable methods and devices are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a Kaplin-Meyer representation for nsPEF conditions tested in treated mice.

FIG. 2 is ultrasound images of tumors in control and treated mice.

FIG. 3 is images of the haematoxylin and eosin (H&E) stained tissue slices of treated and control mice.

FIG. 4. is a plot showing a statistical analysis of the nuclear area in treated mice of FIG. 3.

FIG. 5 is images showing TUNEL staining of select tumors of treated mice.

FIG. 6 is a plot showing statistical analysis of the TUNEL staining of FIG. 5.

FIG. 7 is images showing caspase activation in situ in select tumors of treated mice,

FIG. 8 is a plot showing statistical analysis of active caspase in the tumors of FIG. 7.

FIG. 9 is images showing caspase activation in vivo in select tumors of treated mice.

FIG. 10 is a plot showing statistical analysis of VEGF expression in select tumors of treated mice.

FIG. 11 is a plot showing statistical analysis of CD34 expression in select tumors of treated mice.

### DETAILED DESCRIPTION

Described herein are methods and devices designed to eliminate and/or ablate cancer or other abnormal growths of cells or tissues or eliminate and/or ablate cells or tissue with abnormal functions. The methods and devices induce natural cell death or organic cell death that is used as a normal function to eliminate unneeded or damaged cells in all eukaryotes. The method induces all types of programmed cell death, which can be defined as, but not limited to, caspase-dependent and caspase-independent apoptosis, autophagy, programmed necrosis, which is calpain and/or cathepsin-dependent or calpain and/or cathepsin-independent and cornification. Other atypical cell death modalities induced by this method include, but are not limited to, mitotic catastrophe, anoikis, excitotoxicity, paraptosis, pyroptosis, pyronecrosis, entosis and Wallerian degeneration. Types of typical and atypical programmed cell death are described in Kromer et al., 2009, Classification of cell Death, Cell death and Differentiation 16, 3-11 (doi: 10.1038/cdd.2008.150). The methods and devices can be used to kill tumors percutaneously or internally using endoscopy, for example.

Methods of using pulsed electric fields for therapeutic applications are described in U.S. Pat. No. 6,326,177, issued Dec. 4, 2001, the entirety of which is incorporated herein by reference. The methods and devices described herein use sub-microsecond pulsed electric fields (nsPEFs) to induce different forms of programmed cell death, depending on the nsPEF condition, the stage of the disease, and the cell or tissue type that carries the disease. The methods and devices described herein can also make the individual resistant to the cancer type that was treated and possibly resistant to other cancers. In the examples described herein, resistance to tumor growth was shown to be local. In other words, after the successful treatment of a murine HCC in one flank of mice (6 out of 8 mice), a second injection of tumor cells on the opposite flank did not grow (6 out of 6 mice). In contrast, naïve, age-matched mice readily grew tumors (8 out of 8 mice).

The methods can include applying sub-microsecond electric pulses with electric fields from 10 kV/cm to 500 kV/cm to targeted cells. The sub-microsecond electric pulses can include durations from 1 ns to 999 ns. The application can include a single treatment or can be repeated with repetition rates from 0.1 per second (0.1 Hz) to 10,000 per second (10,000 Hz). For example, the methods can include applying from 1 to 500 pulses with repetition rates from 0.1 per second (0.1 Hz) to 10,000 per second (10,000 Hz). The devices can include pulse power devices that generate electric pulses in accordance with the methods described herein.

The methods and devices are designed to kill cancer cell types and tumors either percutaneous or internally using endoscopy. The targeted cancer cells can include all known types of cancer and abnormal growth in all part of the body.

The methods and devices can also include administration of an immune system booster to improve resistance to the recurrence of new cancer growth. Any suitable adjuvant or immune system booster could be employed. For example, the methods can include administering long pulses and a gene encoding (via electroporation) that encodes a protein which boosts the immune system. The addition of such an immune system booster would be expected to increase the threshold of the immune system of the subject to fight any residual cancer cells.

### EXAMPLES

The present invention is further illustrated by the following specific examples. The examples are provided for illustration only and should not be construed as limiting the scope of the invention in any way.

### Example 1

### Nanosecond Pulsed Electric Field Treatment of Hep1-6 HCC Provides Host Cell Immunity in C57Bl/6 Mice

Table 1 describes the results from an experiment in which NsPEF treatment as described herein provided host resistance to HCC in C57Bl/6 mice. Sixteen one-month old mice having hepatocellular carcinoma (HCC) were divided randomly into an untreated, control group of 8 and a treated group of 8. The HCC tumors were initiated in all mice with 1×10⁶ cells in all mice. When the tumors reached 0.4 cm, the control group was sham treated and the other group was treated with 900 pulses at 100 ns and 55 kV/cm. In 6 of the 8 mice, tumors were eliminated, while the other two mice and the entire control group were euthanized according to an IACUC protocol (2 cm). When the six successfully treated mice were tumor free for 60 days, tumors were initiated in the opposite flank as before. None of these animals grew tumors for 49 days before the experiment was terminated. The two treated mice that were not tumor free for 60 days survived to day 50, while the control, untreated mice were euthanized for tumor burden on day 14-1.7.

TABLE 1

| | # of | Tumor | | | | Growth | Tumor |
|---|---|---|---|---|---|---|---|
| | Mice | diameter | 1^{st} | Survival | Survival | after 2^{nd} | free |
| Group | (1 Mo. old) | at 1^{st} Treatment | Treatment | ratio | days | injection | (days) |
| Treated | 8 | 0.4 cm | 100 ns | 6/8 | 60 | 0/6 | 49 |
| | | | 55 kV/cm | | | | |
| | | | 900 pulse | | | | |
| Control | 8 | 0.4 cm | No nsPEF | 0/8 | 13 ± 5.3 | — | — |

Table 2 shows the time required for tumors to grow to a treatable size of 0.4 cm in mice of different ages. In mice at ages of 1, 2 and 5 months, HCC tumors grew to treatable sizes of 7-9 days for the youngest mice and 14-18 days for 5 month old mice. When mice reached 7 months, no tumors grew in any of the 8 mice in the group,

TABLE 2

| | |
|---|---|
| 1 month (16 mice) | Tumor growth: 7-9 days |
| 2 month (62 mice) | Tumor growth: 7-12 days |
| 5 month (8 mice) | Tumor growth 14-18 days |
| 7 month (8 mice) | No tumor grow up |

These results demonstrate that NsPEF treatment of the Hep1-6 murine HCC provided host resistance to HCC in C57Bl/6 mice. Treating mice with this protocol eliminated the cancer and prevented further cancer growth, i.e., the mice developed immunity against the cancer after one treatment.

### Example 2

### NsPEF Parameter Investigation

Ectopic Hep1-6 HCC in the flanks of the mouse HCC model with 6-8 mice in each group were treated. The following treatment conditions were tested:
(1) Control: no nsPEF treatment (n=8).
(2) 30 ns-R: 100 pulses, 30 ns, 68 kV/cm, three times on alternate days, ring electrode, (n=7)
(3) 30 ns-N: 100 pulses, 30 ns, 68 kV/cm, three times on alternate days, needle array electrode (n=8).
(4) 100 ns-H: 100 pulses, 100 ns, 68 kV/cm, three times on alternate days, needle array electrode, (n=6).
(5) 100 ns-M: 100 pulses, 100 ns 50 kV/cm, three times on alternate days, a needle array electrode (n=6).
(6) 100 ns-L: 100 pulses, 100 ns, 33 kV/cm, three times on alternate days, needle array electrode (n=6).
(7) S100 ns: 900 pulses, 100 ns, 68 kV/cm, single treatment, needle array electrode (n=6).

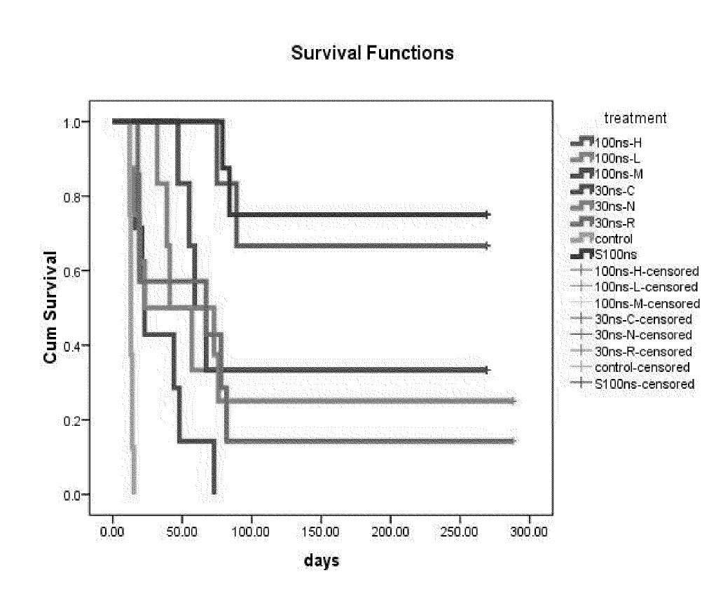

FIG. 1 shows Kaplin-Meyer representations for a number of nsPEF conditions that were used to treat the ectopic Hep1-6 HCC. The specific treatment combinations are indicated in paragraph [0032]. Both ring (R) and needle (N) electrodes were used, treatment regimens included low (L, 33 kV/cm), medium (M, 50 kV/cm) and high (H, 68 kV/cm) electric fields with 30 ns and 100 ns durations, and treatment sessions included three treatments on alternate days as well as a single treatment. The three day regimen included 300 pulses at 30 or 100 ns each day and the single treatment consisted of 900 pulses at 100 ns and 68 kV/cm.

Untreated mice survived for 12-17 days before the tumor burden (2 cm) required euthanasia according to the IACUC protocol of Old Dominion University. In general, treatments fell into two major efficacy zones—greater than 75% survival or less than 40% survival for 260-280 days after treatment. The most effective treatments included the single treatment regime (900 pulses at 100 ns and 68 kV/cm) and three treatments with 300 pulses at 100 ns and 68 kV/cm on each of three alternate days. Both of these conditions used the needle electrode. Less effective treatment included three day treatments with 300 pulses at 30 ns and 68 kV/cm with no real differences with the ring and needle electrode. Less effective were the three treatments with 300 pulses at 100 ns and 33 or 50 kV/cm. For the most effective treatments, high electric fields were required at 100 us with 900 pulses either in a single session lasting 15 minutes or accumulated over three treatment days.

### Example 3

### Tumor Growth Measurement

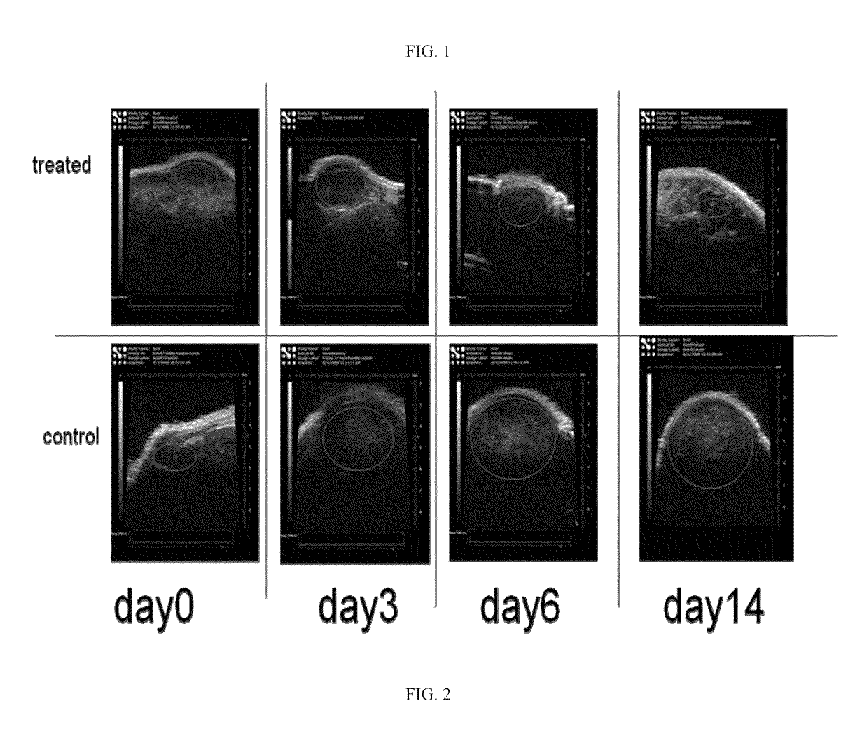

Mice were treated with 300 pulses at 100 ns and 68 kV/cm, three times on alternate days with needle array electrode, with 100 pulses at 30 ns and 68 kV/cm, three times on alternate days with needle array electrode, or not treated. Tumors were imaged daily using an ultrasound (Visualsonics Vevo 770, Visualsonics Inc., Toronto) with model 708 scan head at 55 MHz. Referring to FIG. 2, days 0, 3, 6 and 14 are shown. Tumor dimensions and structure were recorded after the tumor injection and followed up post nsPEFs treatment. Tumor length and width were measured daily by using a Vernier caliper. Tumor volume was calculated by O'Reilly's equation: V(volume)=(tumor width)²×(tumor length)×0.52.

FIG. 2 shows tumor growth visualized with ultrasound in control and treated mice with three treatments on alternate days beginning on day 0 with 100 pulses at 68 kV/cm with 30 ns or 100 ns durations over a 14 day period. Treatment began when tumors were about 0.4 cm. Tumors disappeared with the 100 ns pulse to nearly non-detectable levels 14-21 days after the first treatment in 6 of 8 mice. A small mass of remaining pigment made it difficult to determine when the tumor was completely eliminated. For 30 ns treatments, tumor regression was slower and was only effective in 25% of mice.

### Example 4

### Short Term Morphology Changes after nsPEF Treatment and Statistical Analysis of Nuclear Area

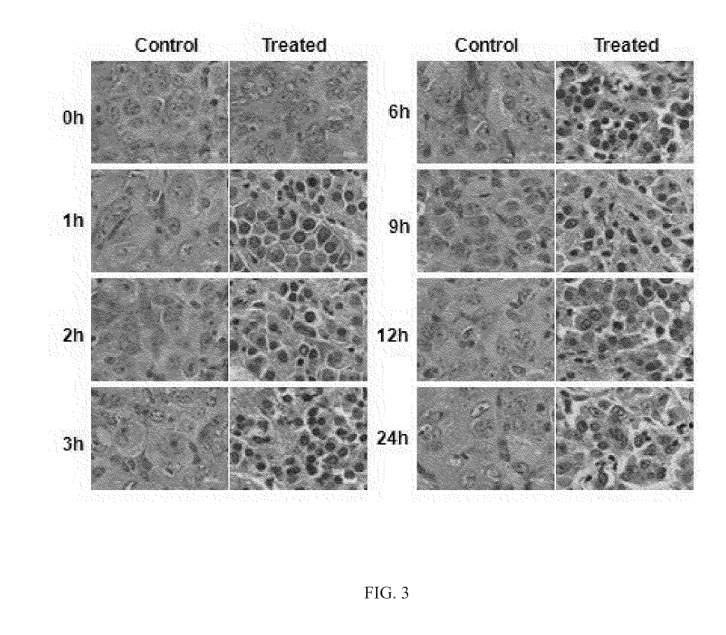

Referring to the results shown in FIG. 3, eight mice were treated by 300 pulses of 100 nsPEFs with a needle array electrode at 68 kV/cm. Mice were euthanized at 0-24 hours as indicated after nsPEF treatment for tumor histological analysis. Tissue slices were stained with H&E at each time point and shown as control in left panels and treated in right panels of FIG. 3.

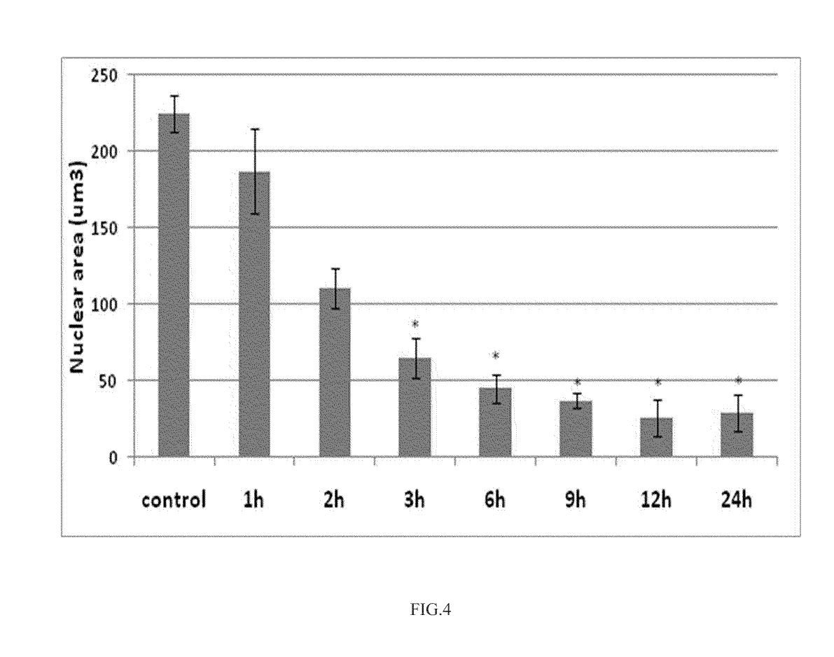

FIGS. 3 and 4 illustrate effects on short-term tumor morphology (FIG. 3) with focus on nuclear area (FIG. 4) after a single treatment with 300 pulses at 100 ns and 68 kV/cm using the needle electrodes. FIG. 4 is a statistical analysis of the nuclear area. Referring to the results shown in FIG. 4, under conditions described in paragraph [0037], 100 nuclei were randomly selected and outlined in ten non-overlapping fields of each section at 200× magnification. The nuclear area was calculated by MATLAB software and summed as the mean±SD.

At 1, 2, 3, 6, 9, 12 and 24 hours after treatment two tumors from each mouse were removed and paraffin imbedded for histological analysis. Sections were stained with H&E and assessed microscopically for abnormal cell morphology. In FIG. 3, H&E staining revealed Hep1-6 tumor ultra-structure and nuclear changes after treatment. Tumor cells featured clear and regular nuclei with prominent nucleoli. The cytoplasm was characteristically purple and homogeneous. The nucleus were round, light blue stained with nucleoli. Treated tumor nuclei dramatically shrank and condensed. The tumor cell connections broke down, losing the cord-like supporting structure on which tumor cells extend. Individual cells became multi-angular with decreased nuclear/plasma ratios. The tumor connection and pattern became unclear and disordered.

### Example 5

### Effect of nsPEF on TUNEL Staining

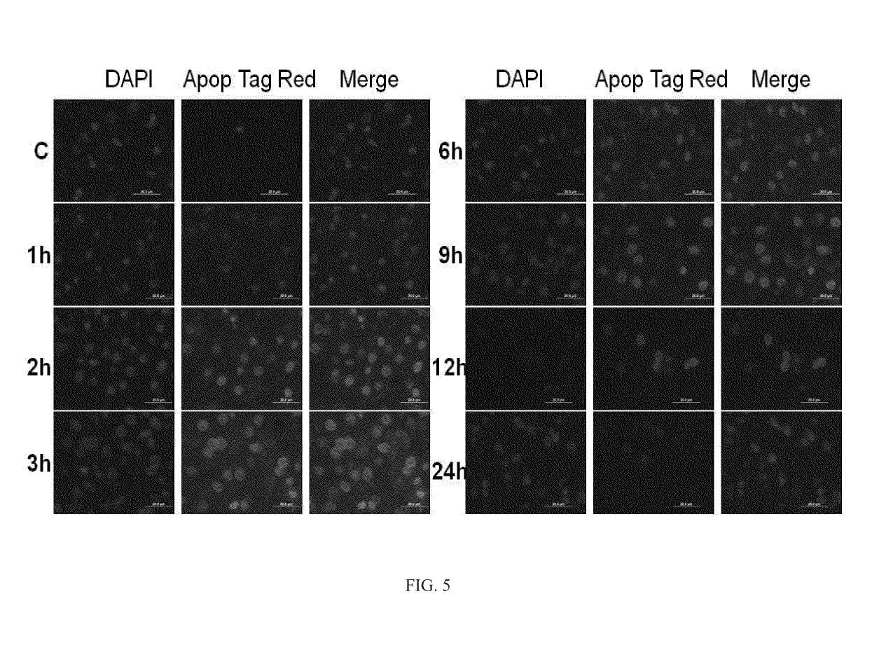

Eight mice were treated with 300 pulses at 100 ns and 68 kV/cm with a needle array electrode. Two tumors on each mouse were selected randomly for control or pulse treatment. Mice were euthanized at 0-24 hours after nsPEF treatment as indicated and prepared for TUNEL analysis in situ using Apot Tag Red (FIG. 5, middle panel). Nuclei were stained with DAPI (FIG. 5, left panel). Merged images were created (FIG. 5, right panel).

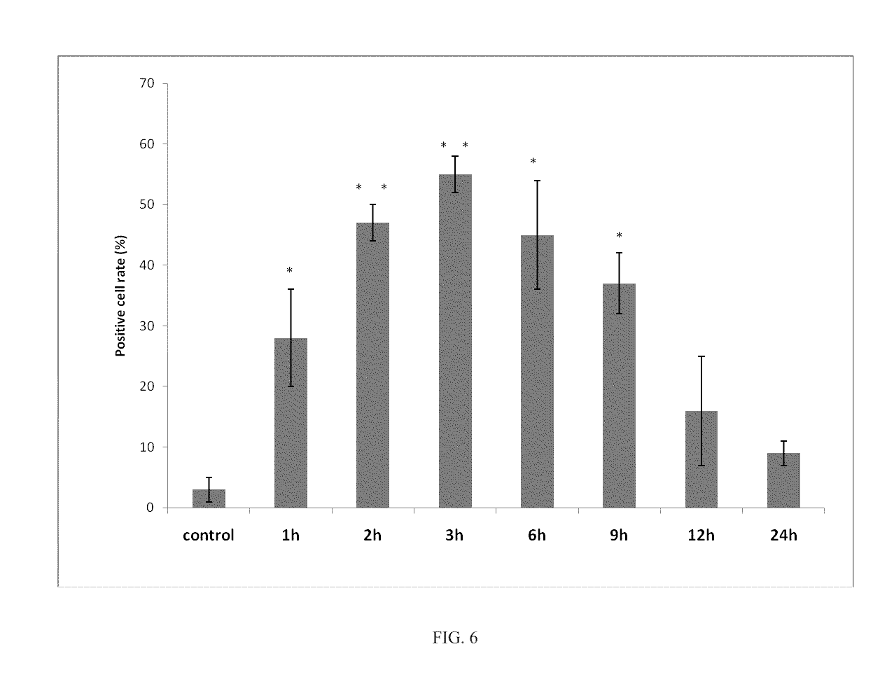

FIG. 6 is a statistical analysis of the TUNEL staining For conditions described in paragraph [0040], 100 nuclei were randomly selected and outlined in ten non-overlapping fields of each section at 200× magnification. Positive cells were outlined and counted by software Image J and then summed as the mean±SD.

FIGS. 5 and 6 analyze treated tumor nuclei using TUNEL to indicate oligonucleosomal DNA fragmentation as a marker for DNA damage and as an apoptosis marker. Two tumors from each mouse were selected randomly for control or nsPEF treatment and paraffin imbedded for TUNEL analysis in situ using Apo Tag Red. In FIG. 5, fluorescent microscopy showed tumor cell nuclei stained bluish-purple with DAPI and cells undergoing apoptosis as reddish orange cytoplasmic halos as TUNEL positive. The merged images revealed apoptotic cells with pinkish nuclei and non-apoptosis cells as purple cells.

FIG. 6 shows a quantitative analysis as the percentage of cells with apoptotic nuclei. One hundred nuclei were randomly selected and outlined in ten non-overlapping fields of each section at 200× magnification. Positive cells were outlined and counted by software Image J and then summed as the mean±SD. The percentage of apoptotic cells increased from 1 h to 9 h significantly after nsPEF treatment versus control tumors (P<0.05). The peak of apoptotic nuclei was about 3 h after nsPEF treatment.

### Example 6

### Effect of nsPEFs on Caspase Activation In Situ

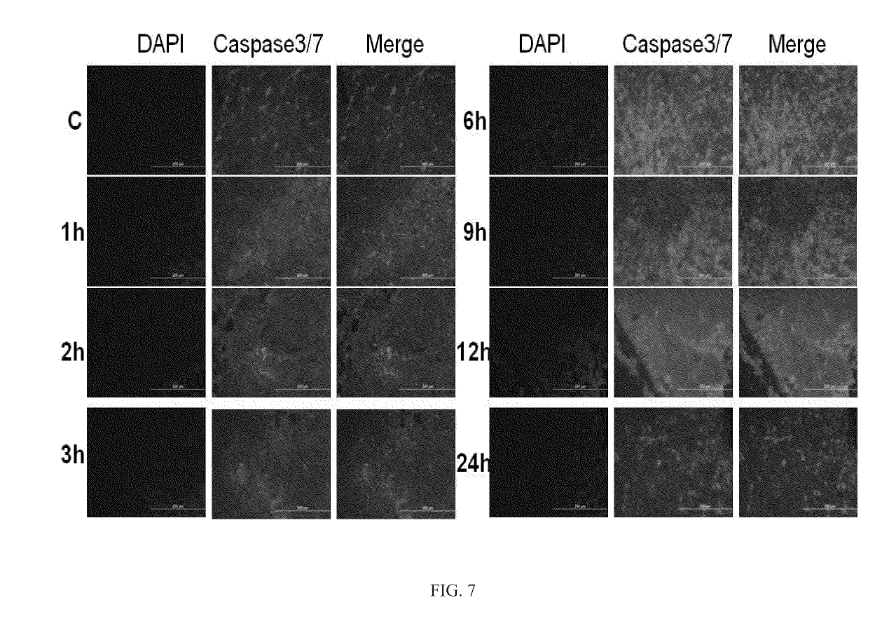

Eight mice were treated with 300 pulses at 100 ns and 68 kV/cm with a needle array electrode. Two tumors on each mouse were selected randomly for control or pulse treatment. Mice were euthanized at 0-24 hours after nsPEF treatment as indicated, and tumors from each mouse were removed and prepared for analysis of the presence of active caspases using antibodies specific for active caspase-3 and -7. The secondary antibody was label with Alexa Fluor-488 (green) (FIG. 7, middle panels). Nuclei were stained with DAPI (blue) (FIG. 7, left panels). Merged images were created showing cells with active caspase-3/7 as an aqua shade (FIG. 7, right panels).

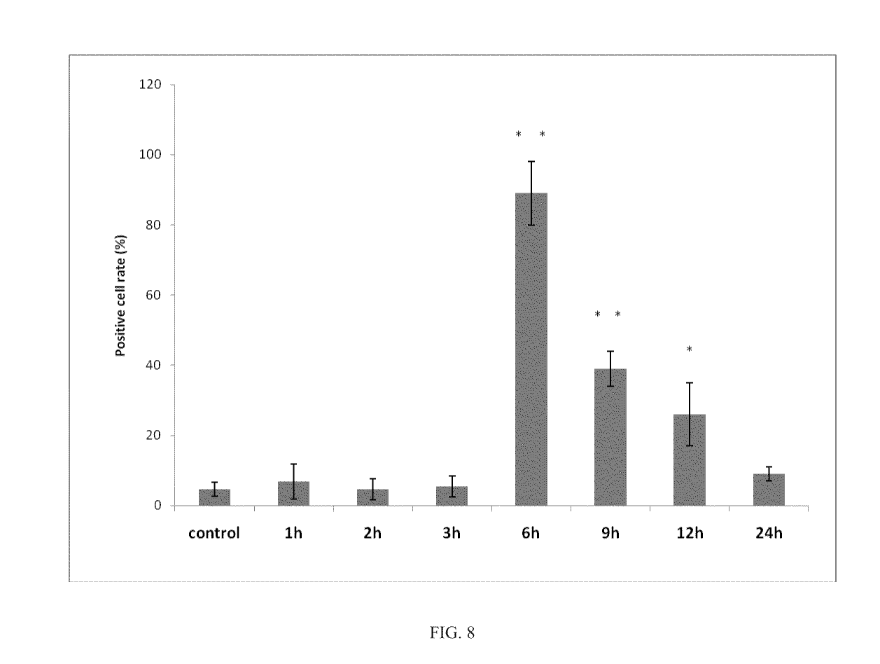

FIGS. 7 and 8 analyze the presence of active executioner caspases-3 and -7 using antibodies specific for the respective active enzymes. Two tumors from each mouse were selected randomly for control or nsPEF treatment. At 1, 2, 3, 6, 9, 12, and 24 hours after treatment, two tumors from each mouse were removed and paraffin imbedded for analysis of the presence of active caspases after a single treatment with 300 pulses at 100 ns and 68 kV/cm using the needle electrodes. In FIG. 7, cell nuclei are stained blue with DAPI (left panels) and cells with active caspases with green fluorescence (middle panels). The merged images show cells with active caspase 3/7 as an aqua shade (right panels).

FIG. 8 is a statistical analysis of active caspase-3 and -7. Conditions as described in paragraph [0044] were used. The number of positive cells was scored by manually counting three sets of at least 100 cells under the microscope. Each experiment was performed twice. Statistical significance is at p<0.05.

FIG. 8 shows a quantitative analysis of percentages of cells with active caspases. The statistical analysis showed percentages of caspase 3 and 7 activation did not significantly increase until 6 h to 12 h after nsPEF treatment versus control tumors.(P<0.05). The peak of active caspases was about 6 h after nsPEFs.

### Example 7

### Effect of nsPEFs on Active Caspase In Vivo

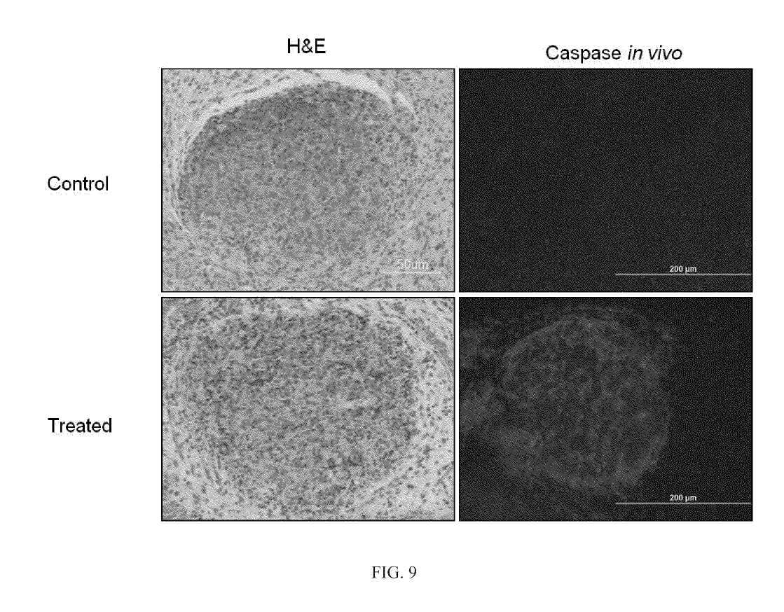

Referring to the results shown in FIG. 9, four mice were treated with 300 pulses at 100 ns and 55 kV/cm with a needle array electrode. Two tumors on each mouse were selected randomly for control or pulse treatment. Six hours after nsPEF treatment of FLIVO (FAM-VAD-FMK, green fluorescence) was injected into the internal jugular vein. Thirty (30) minutes later the mice were euthanized, tumor removed snap frozen in liquid nitrogen and tissue sections were prepared for green fluorescent microscopy for active caspases (FIG. 9, right panels). Other slices were prepared for H&E staining (FIG. 9, left panels).

in order to determine the presence of active caspases in vivo FAM-FLIVO green immunofluorescence was used to label cells with active caspases with FAM-VAD-FMK a cell permeable irreversible pan-caspase inhibitor. Four mice were treated by 300 pulses of 100 nsPEFs with a needle electrode at 55 kV/cm. Two tumors on each mouse were selected randomly for control or pulse treatment. Six hours after treatment 50 μl of FLIVO (FAM-VAD-FMK, green fluorescence) was injected into the internal jugular vein. Thirty (30) minutes later the mice were euthanized, tumors removed and snap frozen in liquid nitrogen and tissue sections prepared for fluorescent microscope form active caspases (right panels). Other slices were prepared for H&E staining (left panels of FIG. 9).

FIG. 9 analyzes the effect of nsPEFs on active caspase in vivo. In the H&E stained slides, control tumors showed aggressive growth bounded by a thin fibrous capsule with internal fibrous structure. No active caspase (FLIVO) was detected in the control tumor. In nsPEF treated tumors 6 h post pulse, cells were condensed and detached from the tumor connective tissue. Active caspases (FLIVO) were detected throughout the whole tumor demonstrating caspase activation in vivo after nsPEF treatment.

### Example 8

### Effect of nsPEFs on Active Caspase In Vivo, on VEGF Expression, and on CD34 Expression

NsPEFs have been shown to have effects on tumors vasculature. The effects on vascular endothelial cell growth factor (VEGF), the most ubiquitous pro-angiogenic factor and a downstream VEGF respondent CD34, a common endothelial micro-vessel density (MVD) marker were tested.

Four mice were treated with 100 pulses at 100 ns and 68 kV/cm with a needle array electrode and repeated 3 times on alternate days. Another 4 mice with a control tumor in each one were set up separately. Two tumors on each mouse were selected randomly for control or pulse treatment. Mice were euthanized on days 0 (control), 7, 14 and 21 after nsPEF treatment and tumors were removed and prepared for immunohistochemistry (MC) with antibodies to VEGF and CD34. For the effect of nsPEF on VEGF, tissue slices were incubated with an antibody to VEGF. The appearance of VEGF was indicated by brown color after staining with diaminobenzidine.

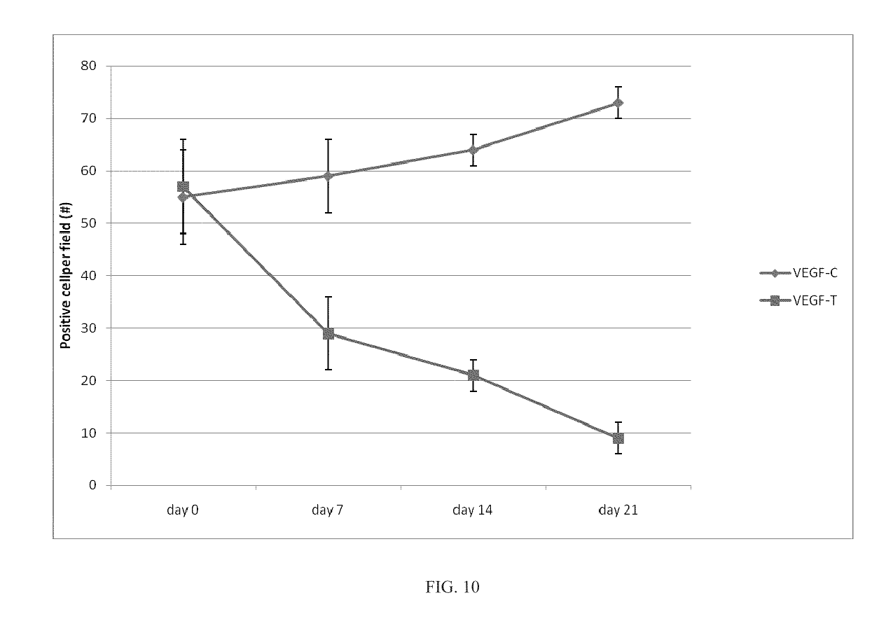

The IHC staining with brown cells demonstrated the presence of VEGF and FIG. 10 shows a quantitative analysis of the results. Conditions were the same as those described in paragraph [0052]. For each time point, there was one mouse. For every sample 3 slides were stained by IHC. The IHC staining outlined the micro vessels in Hep1-6 tumors. The brown vessels were counted and summarized as the mean±SD based on 3 slides from the same mouse at each time point. Statistical significance is at p<0.05. In control tumors, VEGF positive cells increased nearly linearly over the three week period of analysis. In contrast, treated tumors showed an 83% decrease in VEGF compared to the day of treatment and a 7-fold decrease compared to the untreated control on day 21.

For the effect of nsPEF on CD34 expression, the nsPEF conditions and preparation for immunohistochemistry described in paragraph [0052] were used, except antibodies to CD34 were used.

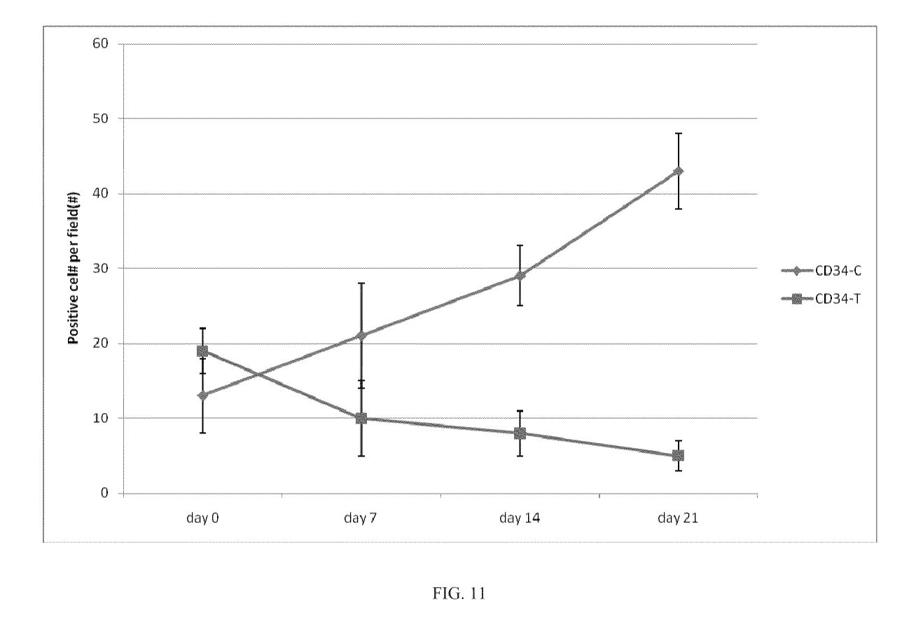

IHC staining with brown cells demonstrated the presence of CD34 and FIG. 11 shows a quantitative analysis of the results. The nsPEF conditions described in paragraph [0053] were used. In untreated controls CD34 increased more than 4-fold after 3 weeks. In contrast, CD34 decreased 75% from the day of treatment and more than 8-fold less that the untreated control on day 21.

### Other Embodiments

Any improvement may be made in part or all of the compositions, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method for treatment of cancer comprising:
applying a sub-microsecond electric pulse to cancer cells,
wherein the sub-microsecond electric pulse comprises an electric field ranging from 10 kV/cm to 500 kV/cm.

2. The method according to claim 1, wherein the duration of the sub-microsecond pulse ranges from 1 nanosecond to 999 nanoseconds.

3. The method according to claim 1 further comprising:
repeating the applying step with repetition rates ranging from 0.1 per second (0.1 Hz) to 10,000 per second (10,000 Hz).

4. The method according to claim 3, wherein up to 5000 sub-microsecond electric pulses are applied.

5. The method according to claim 1 further comprising administering an immune system booster.

6. A device for the treatment of cancer comprising:
a generator which provides sub-microsecond electric pulses,
wherein the sub-microsecond electric pulses comprise electric fields ranging from 10 kV/cm to 500 kV/cm.

7. The device according to claim 6, wherein the generator provides the sub-microsecond electric pulses with repetition rates ranging from 0.1 per second (0.1 Hz) to 10,000 per second (10,000 Hz).
